(19) United States
(12) Patent Application Publication
(10) Pub. No.: US 2011/0275161 A1
(43) Pub. Date: Nov. 10, 2011

(54) METHOD OF DETECTING NERVE AGENTS USING NOVEL ASSAY AGENTS

(75) Inventors: SHOUFA HAN, San Bruno, CA (US); Changping Shi, San Francisco, CA (US)

(73) Assignee: MCLAB, South San Francisco, CA (US)

(21) Appl. No.: 12/774,695

(22) Filed: May 5, 2010

(51) Int. Cl. G01N 21/76; C07D 491/22; C07D 491/107

(52) U.S. Cl. 436/104; 548/410; 546/15; 436/172

(57) ABSTRACT

The teachings provided herein are directed to compounds and methods for detecting nerve agents using rhodamine-derived detection compounds. A rhodamine-derived detection compound for nerve agents can include any rhodamine derivative that can bind to a nerve agent and produce a detectable signal for detection of the nerve agent. In some embodiments, the rhodamine-derived detection compound comprises rhodamine B-hydroxamate and the nerve agent can be detected in amounts as low as about 10 ppm.

### BACKGROUND

1. Field of the Invention

The teachings provided herein are directed to compounds and methods for detecting nerve agents using a rhodamine-derived detection compound.

2. Description of the Related Art

Nerve agents are an increasing homeland security concern due to the horrifying effects that could be realized from a terrorist attack. Moreover, increased concerns over pollution, such as the increased use of pesticides that are considered nerve agents, has created hazards to some world communities. The utility of nerve agents stems from their extraordinary toxicity; a lethal dose can be as little as 0.01 mg of the nerve agent per 1.0 kg of a man's body weight, or 0.70 mg for an average 70 kg man. As such, improved detection compounds and sensitive methods of detecting nerve agents will be desirable to both government officials and the general public.

There are a wide variety of nerve agents including, for example, G-series nerve agents, V-series nerve agents, and Novichok nerve agents. Sarin is an example of a G-series nerve agent and is an ester of methyl phosphonic acid. Such an ester of methyl phosphonic acid has a moderately good leaving group as the third substitution. As such, this nerve agent, for example, can irreversibly modify the active site serine of acetylcholine esterases and lead to a complete loss of enzyme activity.

Conventional detection systems suffer a number of problems. For example, enzyme/antibody-based methods suffer from reagent storage and stability issues. Instrument-based methods, such as mass spectrometry, suffer from too much selectivity, lack of sensitivity, lack of portability, and high cost of equipment. To overcome these limitations, fluorogenic or chromogenic chemosensors have been investigated.

One of skill will appreciate that rhodamine compounds have been modified for a variety of uses that included the detection of chemicals and tracing methods. “A rhodamine compound” is one of a family of related chemical compounds, sometimes referred to as fluorone dyes. Examples of rhodamine compounds include rhodamine 6G and rhodamine B. As a tracer dye, rhodamine compounds have been used within water to determine the rate and direction of flow and transport of a material. The fluorescence of the rhodamine dyes can be detected using a fluorometer. Other examples of known uses rhodamine compounds include biotechnology applications such as fluorescence microscopy, flow cytometry, fluorescence correlation spectroscopy and ELISA.

Accordingly, rhodamine-derived detection compounds that provide an improved detection method for nerve agents will be desirable to both government officials and the general public, as well as appreciated by those skilled in the art. One of skill will appreciate the compounds and methods of using the compounds taught herein, which provide (i) low selectivity, capable of sensing nerve gases in general rather than particular gases; (ii) a high sensitivity, (iii) portability, and (iv) a comparatively low cost of equipment.

### SUMMARY

The teachings provided herein are directed to compounds and methods for detecting nerve agents using a rhodamine-derived detection compound. In some embodiments, the teachings are directed to a rhodamine-derived detection compound for nerve agents comprising the structure:

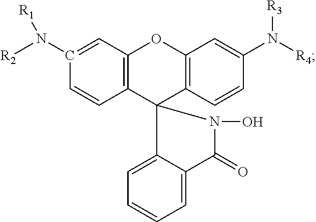

or, a precursor thereof.

In these embodiments, the R₁-R₄ are each independently selected from the group consisting of straight-chain or branched alkylenes having up to 6 carbons and optionally cyclizing with an aromatic host ring, and the compound can react with a nerve agent to form a fluorescent product for detection of the nerve agent at concentrations below 100 ppm.

In some embodiments, the R₁-R₄ are each independently selected from the group consisting of methylene, ethylene, and propylene. And, in some embodiments, the compound comprises rhodamine B-hydroxamate. In fact, in some embodiments, the detection compound can react with a nerve agent to form a fluorescent product for detection of the nerve agent at concentrations as low as about 10 ppm.

In many embodiments, the nerve agent can be any nerve agent that can bind with the detection compound and form a fluorescent product. For example, the nerve agent can comprise an organophosphate. In some embodiments, the nerve agent is a G-series nerve agent, a V-series nerve agent, or a Novichok nerve agent. In some embodiments, the nerve agent can be selected from the group consisting of Sarin, Cyclosarin, Soman, Vx, and Tabun.

The teachings provided herein allow the production of a wide variety of rhodamine-derived detection compounds. In some embodiments, the compound can be selected from the group consisting of:

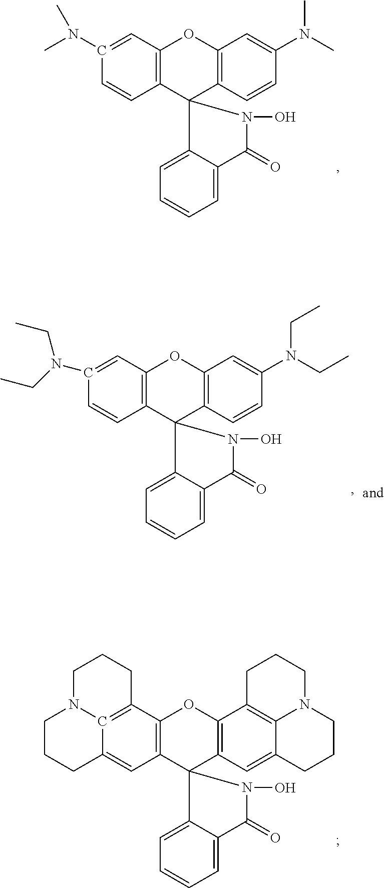

or a precursor thereof.

Accordingly, the teachings are directed to a method of detecting a nerve agent, comprising reacting the detection compounds described above with a nerve agent, such as those also described above. After the reaction product is formed, the method proceeds by detecting the product formed by the reaction. As described above, the detection compound can react with the nerve agent in some embodiments to form a fluorescent product for detection of the nerve agent at concentrations below 100 ppm. And, in some embodiments, the detection compound reacts with the nerve agent to form a fluorescent product for detection of the nerve agent at concentrations as low as about 10 ppm.

### BRIEF DESCRIPTION OF THE FIGURES

FIG. 1 shows highly detectable fluorescence, as well as color development visible to the naked eye, that can be realized as a result of the reaction between the rhodamine-hydroxamate derivatives and the nerve agent, according to some embodiments.

FIG. 2 illustrates a highly detectable fluorescence emission spectra of the products generated from diethyl chlorophosphate with rhodamine B-hydroxamate (1 mg/mL) in the presence of 3% (v/v) triethylamine in DMF, according to some embodiments.

FIG. 3 illustrates UV-Vis absorption spectra of products generated from diethyl chlorophosphate with rhodamine B-hydroxamate (1 mg/mL) in the presence of 3% (v/v) triethylamine in DMF, according to some embodiments.

FIG. 4 illustrates how the sensor can be used (a) in solution, (b) mixed with a solid, or (c) covalently linked to a solid, according to some embodiments.

### DETAILED DESCRIPTION OF THE INVENTION

As described above, the teachings provided herein are directed to compounds and methods for detecting nerve agents using rhodamine-derived detection compounds.

In some embodiments, the teachings are directed to a class of hydroxamate-based chemosensors for detection of nerve agents. The term “chemosensor” and “sensor” and “assay agent” can be used interchangeably in some embodiments. Likewise, the term “nerve agent” and “nerve gas” can also be used interchangeably in some embodiments.

The chemosensors can be developed, in some embodiments, through a reaction of hydroxylamine with a carboxylic acid. For example, the carboxyl group of rhodamine can be converted to give a non-fluorescent hydroxamate molecule under basic reaction conditions. While not intending to be bound by any theory or mechanism of action, a proposed reaction mechanism based on rhodamine B is the following:

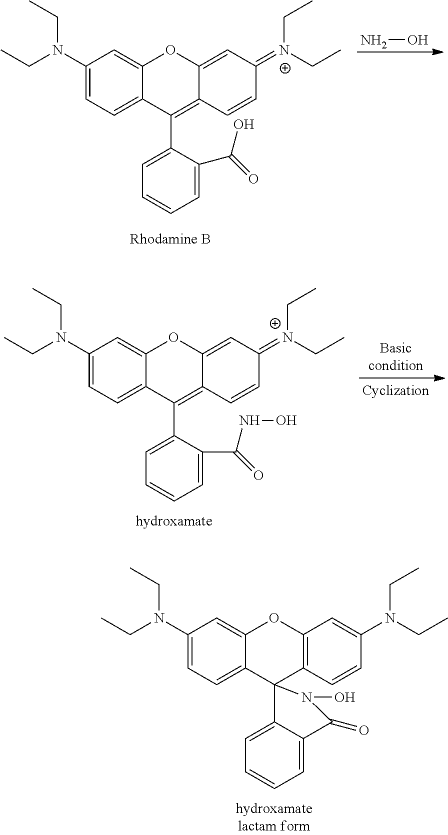

A sensor can be produced, for example, to include the intramolecular 5-membered ring lactam of the hydroxamate, where the nerve gas can have a leaving group, such as a halogen, for example, that is substituted by the nucleophilic sensor. Upon reacting the sensor with a nerve agent mimic having a chlorine leaving group, for example, the sensor serves as a good nucleophile to the chlorine leaving group of the nerve agent mimic, allowing for substitution of the chlorine with the sensor, at the hydroxy group of the hydroxamate.

While not intending to be bound by any theory or mechanism of action, one of skill will appreciate that a possible chemical mechanism, although not fully defined at this time, might include a Lossen rearrangement and intromolecular cyclization, described as follows:

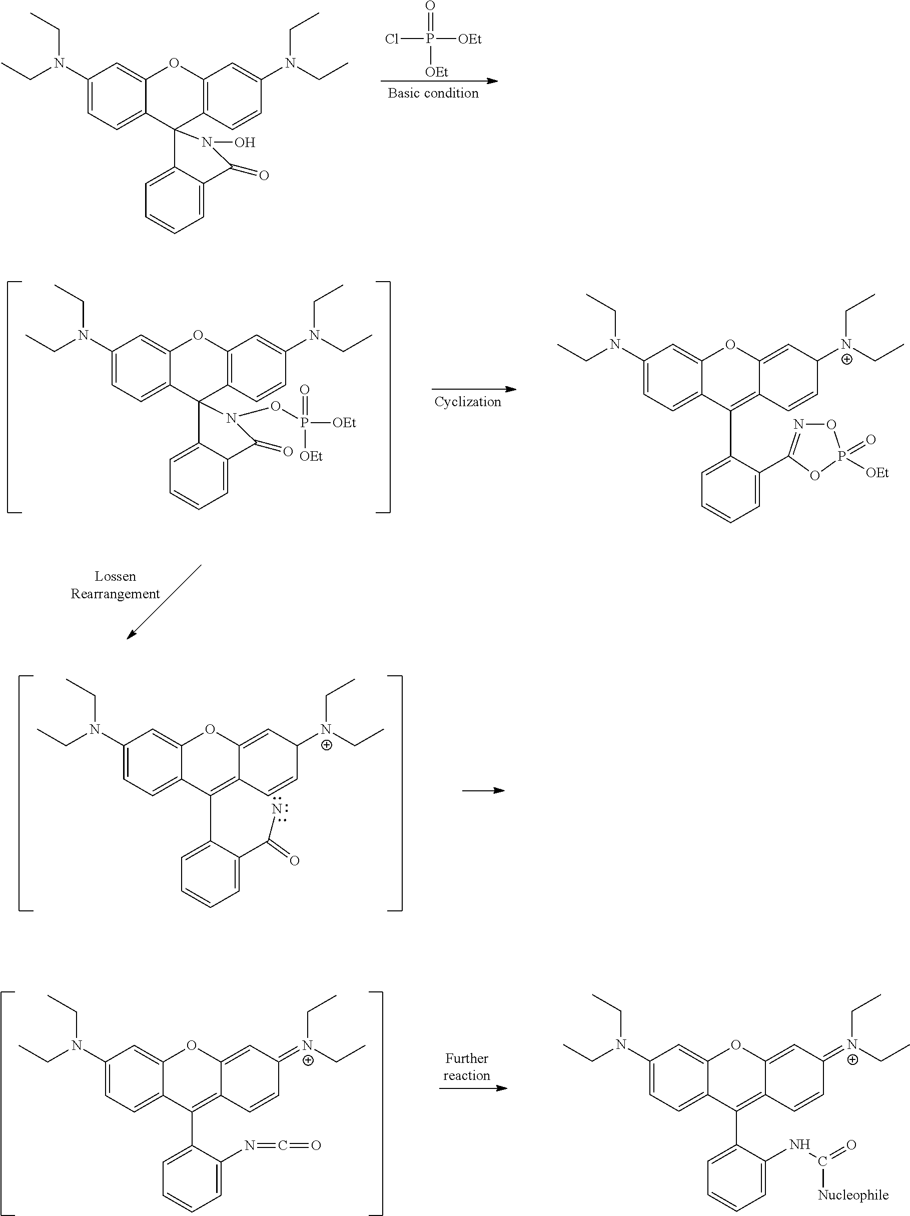

The proposed pathways show products that share a structural similarity, the rhodamine moiety. The rhodamine moiety, possibly formed from a nerve agent promoted opening of the intromolecular lactam, has proven to be colorimetric as well as highly fluorescent. These products facilitate visual detection, instrumental detection, or a combination thereof, in many embodiments.

In some embodiments, the detection of a nerve gas can be accomplished by exposing solutions of the sensor compound in a basic reaction media to a sample expected to contain the nerve gas. The reaction mixtures can be allowed to incubate at a desired time and temperature prior to attempting detection using, for example, colorimetry, fluorescence, or a combination thereof. The colorimetry can be done usual a direct visual observation or an instrument. The fluorescence can be, for example, detected using intensity-emission fluorescence or UV-Vis absorbance. One of skill will appreciate that the quantities can be determined with a high accuracy and precision using standard calibration curve techniques. In some embodiments, rhodamine-hydroxamate was shown to be capable of detecting nerve agent mimics, for example, in a variety of media.

One of skill will appreciate that various reaction conditions can be used in the teachings provided herein. The reaction conditions can include reaction time, temperature, and media.

The reaction media in some embodiments can include DMF, nitrobenzene, DMSO, THF, ethyl acetate, or a combination thereof, for example. In fact, in some embodiments, the reactions may include any solvent which is substantially nonreactive with the reactants, the intermediates, or products at the temperatures at which the reactions are carried out, temperatures which may range, for example, from the solvent's freezing temperature to the solvent's boiling temperature. A given reaction may be carried out in one solvent or a mixture of more than one solvent. And, the solvent can be “substantially non-reactive” when any detected noise from an undesired reaction product does not affect the usefulness of the nerve gas detection to the extent that it is no longer a desirable method to one of skill.

In some embodiments, the solvent may be halogenated. Halogenated solvents can include, for example, chlorobenzene, fluorobenzene or dichloromethane.

In some embodiments, the solvent may be an ether solvent. Ether solvents can include, for example, tetrahydrofuran, diethyl ether, ethylene glycol dimethyl ether, ethylene glycol diethyl ether, diethylene glycol dimethyl ether, diethylene glycol diethyl ether, triethylene glycol dimethyl ether, or t-butyl methyl ether.

In some embodiments, the solvent may be a protic solvent. Protic solvents may include, for example, water, methanol, ethanol, 2-nitroethanol, 2-fluoroethanol, 2,2,2-trifluoroethanol, ethylene glycol, 1-propanol, 2-propanol, 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, benzyl alcohol, phenol, or glycerol.

In some embodiments, the solvent may be an aprotic solvent. Aprotic solvents may include, for example, tetrahydrofuran (THF), dimethylformamide (DMF), dimethylacetamide (DMAC), 1,3-dimethyl-3,4,5,6-tetrahydro-2(1H)-pyrimidinone (DMPU), 1,3-dimethyl-2-imidazolidinone (DMI), N-methylpyrrolidinone (NMP), formamide, N-methylacetamide, N-methylformamide, acetonitrile, dimethyl sulfoxide, propionitrile, ethyl formate, methyl acetate, hexachloroacetone, acetone, ethyl methyl ketone, ethyl acetate, sulfolane, N,N-dimethylpropionamide, tetramethylurea, nitromethane, nitrobenzene, or hexamethylphosphoramide.

In some embodiments, the solvent may be a basic solvent. Basic solvents may include, for example, a 2-, 3-, or 4-picoline, pyrrole, pyrrolidine, morpholine, pyridine, or piperidine.

In some embodiments, the solvent may be a hydrocarbon solvent. Hydrocarbon solvents may include, for example, benzene, cyclohexane, pentane, hexane, toluene, cycloheptane, methylcyclohexane, heptane, ethylbenzene, m-, o-, or p-xylene, octane, indane, nonane, or naphthalene.

A “rhodamine-derived detection compound for nerve agents,” as described herein, can include any rhodamine derivative that can bind to a nerve agent and produce a detectable signal for detection of the nerve agent. In some embodiments, the rhodamine-derived detection compound comprises the structure:

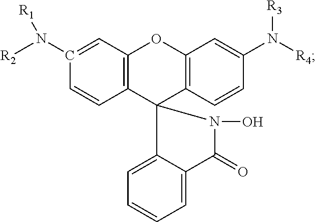

or a precursor thereof.

In some embodiments, the R₁-R₄ can be each independently selected from the group consisting of straight-chain or branched alkylenes having up to 6 carbons and optionally cyclizing with an aromatic host ring, and the compound can react with a nerve agent to form a fluorescent product for detection of the nerve agent at concentrations below 100 ppm. One of skill will appreciate that the term “precursor” can refer to, for example, the acid version of the lactam prior to cyclization into the hydroxamate structure, or any structure known to one of skill that can convert to the rhodamine-hydroxamate type structures using mechanisms readily known to those of skill. As will be appreciated by one of skill, in some embodiments, the sensor can be, or comprise, any nucleophilic moiety having any substituent, other than hydroxy, for example, as long as the nucleophilic strength of the sensor is greater than the nucleophilic strength of the leaving group of the nerve agent. It should be appreciate that, in some embodiments, the nerve agent should sufficiently promote the opening of the lactam of the hydroxamate and form a compound that is detectable, such as by fluorescence or colorimetrics. For example, in some embodiments, the nucleophilic moiety of the rhodamine-lactam-type sensor can be an amino group forming a hydrazide, rather than a hydroxyl group from the hydroxamate. One of skill in the art will appreciate the scope of chemical structures, and the reaction mechanisms, that can be used to form the sensor.

In some embodiments, the R₁-R₄ groups can be each independently selected from the group consisting of methylene, ethylene, and propylene, for example. And, in some embodiments, the compound comprises rhodamine B-hydroxamate. In fact, in some embodiments, the detection compound can react with a nerve agent to form a fluorescent product for detection of the nerve agent at concentrations as low as about 10 ppm. However, it should be appreciated by one of skill that, in some embodiments, the R₁-R₄ groups cannot all be the same, for example, all methylene, all ethylene, all propylene, all butylene, and the like. It should also be appreciated that the R₁-R₄ groups can be selected by one of skill for suitability in particular reaction media, considerations of steric hindrance, and the like.

The methods provided herein can be moderately or highly sensitive to nerve gases. In some embodiments, the methods are highly sensitive and capable of measuring very low levels of nerve gas due to the low background noise produced by the sensor. The low level of noise allows for detection of very low concentrations of nerve gas. For example, a rhodamine B-hydroxamate sensor can have a minimal fluorescence at 590 nm while the products of the reaction of the sensor with the nerve gases can be comparatively highly fluorescent, as well as UV absorbent at a range, for example, of 560-570 nm.

In some embodiments, a nerve agent may be detected in most expected concentrations. For example, a nerve agent may be detected to concentrations ranging from about 0.05 ppm to about 10 ppm, from about 0.05 ppm to about 5 ppm, as low as about 1 ppm, as low as about 10 ppm, as low as about 20 ppm, from about 1.0 ppm to about 10 ppm, from about 1.0 ppm to about 5.0 ppm, greater than 500 ppm, from about 1 ppm to about 500 ppm, from about 10 ppm to about 500 ppm, from about 15 ppm to about 500 ppm, from about 20 ppm to about 500 ppm, from about 30 ppm to about 500 ppm, from about 50 ppm to about 500 ppm, from about 75 ppm to about 500 ppm, and from about 100 ppm to about 500 ppm, or any range therein. In some embodiments, the nerve agent may be detected in an amount as low as about 1 ppm, about 5 ppm, about 10 ppm, about 15 ppm, about 20 ppm, about 25 ppm, about 30 ppm, about 50 ppm, about 75 ppm, about 100 ppm, about 200 ppm, and greater than about 500 ppm, or any range therein.

In many embodiments, the nerve agent can be any nerve agent that can bind with the detection compound and form a fluorescent product. For example, the nerve agent can comprise an organophosphate. In some embodiments, the nerve agent is a G-series nerve agent, a V-series nerve agent, a Novichok nerve agent, or an insecticide. G-Series nerve agents, for example, generally include those discovered and synthesized during or soon after World War II and is the first and oldest family of nerve agents. Examples of G-series nerve agents include, but are not limited to, GA (Tabun), GB (Sarin), GD (Soman), and GF (Cyclosarin).

V-Series nerve agents include a class of organophosphate compounds (organophosphate esters of substituted aminoethanethiols) that were found to be quite effective pesticides. Examples include, but are not limited to, Amiton (VG), VX, VE, VG, and VM. The V-series nerve agents are about 10 times more toxic than Sarin (GB) and are persistent agents, meaning that they do not degrade or wash away easily, can remain on clothes and other surfaces for long periods, and can be used to blanket terrain to guide or curtail the movement of enemy ground forces. V-series nerve agents are oily and are primarily a dermal contact hazard. Novichok nerve agents are a series of organophosphate compounds developed by the Soviet Union as highly deadly chemical weapons unknown to the West, undetectable by standard NATO chemical detection equipment, and able to act despite chemical protective gear.

In some embodiments, pesticides can also be classed as nerve agents. For example, a number of insecticides, the phenothiazines, organophosphates such as dichlorvos, malathion, and parathion, are considered nerve agents. Although the metabolism of insects is different from mammals, these chemicals can create acute toxicity and death through the same mechanism as other nerve agents. Organophosphate pesticide poisoning is often considered a major cause of disability in many developing countries and, in fact, is often used as a method of suicide.

As such, one of skill will appreciate that the detection of many nerve agents is addressed by the teachings provided herein. In some embodiments, the nerve agent can be selected from the group consisting of Sarin, Cyclosarin, Soman, Vx, and Tabun, having the following general structures:

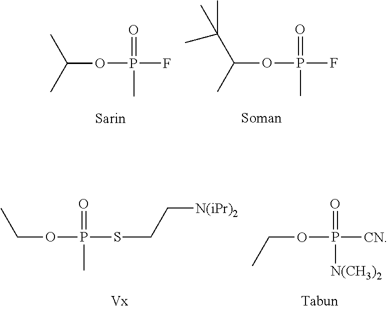

One of skill will also appreciate that a wide variety of rhodamine-derived detection compounds can be produced using the teachings provided herein. In some embodiments, the compound can be selected from the group consisting of:

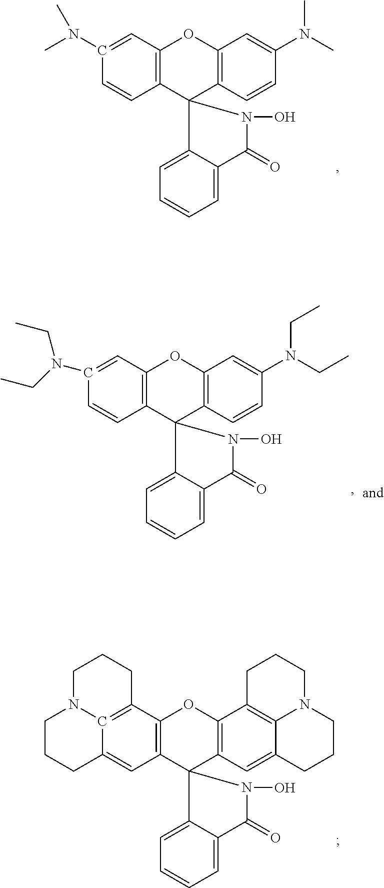

or a precursor thereof.

Accordingly, as can be seen from the above, the teachings are directed to a method of detecting a nerve agent. The method comprises reacting the detection compounds described above with the nerve agent, such as those also described above. After the reaction product is formed, the method proceeds by detecting the product formed by the reaction. As described above, the detection compound can react with the nerve agent in some embodiments to form a fluorescent product for detection of the nerve agent at concentrations below 100 ppm. And, in some embodiments, the detection compound reacts with the nerve agent to form a fluorescent product for detection of the nerve agent at concentrations as low as about 20 ppm.

Without intending to be limited to any theory or mechanism of action, the following examples are provided to further illustrate the teachings presented herein. It should be appreciated that there are several variations contemplated within the skill in the art, and that the examples are not intended to be construed as providing limitations to the claims.

### EXAMPLES

### Example 1

### Production of Rhodamine B-Hydroxamate

In this example, a rhodamine-derived detection compound for nerve agents was produced. 10 g of rhodamine B dissolved in 50 mL of ethanol was added to an aqueous solution (80 mL) containing hydroxylamine-HCl (10 g) and sodium hydroxide (15 g). The mixture was stirred at room temperature for 2 hours, and then the solution was poured into water (300 mL) and extracted with methylene dichloride (300 mL). The organic layer was washed with water again (250 mL) and then dried over sodium sulfate. The organic solution was filtered and then evaporated to afford the desired product of rhodamine B-hydroxamate as a brownish solid in about 80% yield.

### Example 2

### Detection of Nerve Agent Mimics Using a Rhodamine-Derived Detection Compound

In this example, a sensitive fluorogenic/chromogenic detection of nerve agent mimics was performed. The rhodamine-hydroxamate derivatives can react, for example, as follows:

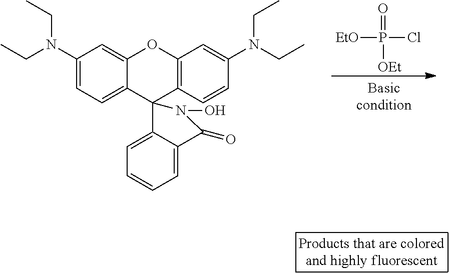

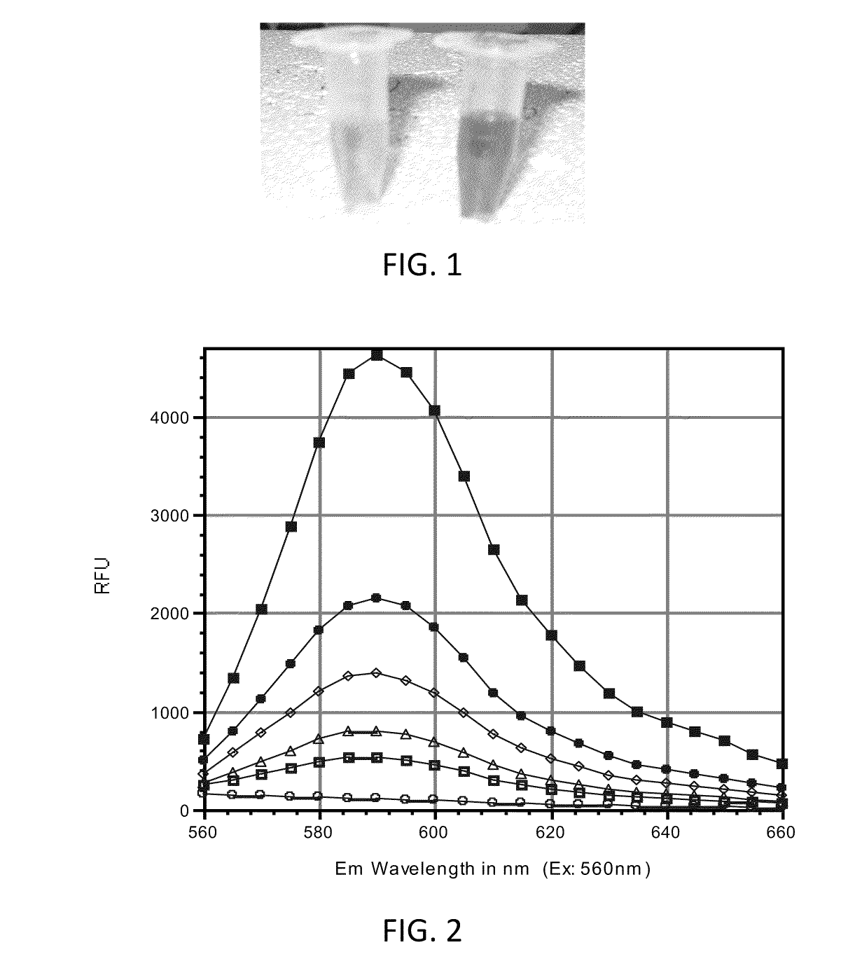

FIG. 1 shows highly detectable fluorescence, as well as color development visible to the naked eye, that can be realized as a result of the reaction between the rhodamine-hydroxamate derivatives and the nerve agent, according to some embodiments. Generally speaking, FIG. 1 is a comparison of rhodamine B-hydroxamate solution (1 mg/mL) in DMF containing triethylamine (3%, v/v) before (left) and after addition diethyl chlorophosphate (500 ppm). In FIG. 1, the fluorescence provides a highly sensitive detection from a reaction product of diethyl chlorophosphate with rhodamine B-hydroxamate in presence of 3% (v/v) of triethylamine in DMF. The yellow tube is the rhodamine B-hydroxamate prior to the reaction, and the reaction products are shown in the right tube, providing a highly detectable fluorescence.

The detection was performed by the addition of 2 uL of diethyl chlorophosphate into solutions of the rhodamine B-hydroxamate (1 mg/mL) in DMF containing 3% (v/v) triethylamine of various volumes (2 mL, 4 mL, 8 mL, 12 mL, 20 mL, 30 mL, 40 mL). The reaction mixtures were incubated at room temperature for about 20 minutes and then the fluorescence was detected using intensity-emission fluorescence or UV-Vis absorbance on Spectramax M5 (by Molecular Devices). One of skill will appreciate that the quantities can be determined, in some embodiments, with a high accuracy and precision using, for example, standard calibration curve techniques.

| | TABLE | | | | | | |
|---|---|---|---|---|---|---|---|
| | NERVE AGENT MIMIC | | | | | | |
| | diethyl chlorophosphate (ppm) | | | | | | |
| INSTRUMENT | 500 | 250 | 170 | 100 | 50 | 25 | 0 |
| Fluorescence Intensity | 4630 | 4043 | 2150 | 1340 | 790 | 510 | 90 |
| measured at 590 nm | | | | | | | |
| (RFU) | | | | | | | |
| UV-Vis absorbance | 3.07 | 1.64 | 1.0 | 0.35 | 0.27 | 0.22 | 0.19 |
| measured at 560 nm | | | | | | | |

Reaction conditions: 2 uL of diethyl chlorophosphate was added into various volume of a DMF solution containing rhodamine B-hydroxamate (1 mg/mL) and triethylamine (3%, v/v) to make a serial diluted reaction mixture with diethyl chlorophosphate at 500 ppm, 250 ppm, 170 ppm, 100 ppm, 50 ppm, and 25 ppm. The reaction mixtures were incubated at room temperature for about 20 minutes, and then the fluorescence was detected using intensity-emission fluorescence or UV-Vis absorbance on a Spectramax M5 (Molecular Devices)

As can been seen, the signal increases as a function of the increase in diethyl chlorophosphate concentration. One of skill will appreciate that, although as low as 25 ppm was clearly identified (510 RFU relative to the background of 90 RFU), much lower concentrations are certainly measurable such, for example, at least 10 ppm in the case of the diethyl chlorophosphate nerve agent mimic that was tested above. UV-Vis was used to confirm the measurements, also proving to be a viable alternative measurement technique in itself.

One of skill will appreciate that useful spectra can be obtained using standard equipment and techniques known to those of skill. FIG. 2 illustrates a highly detectable fluorescence emission spectra of the products generated from diethyl chlorophosphate with rhodamine B-hydroxamate (1 mg/mL) in the presence of 3% (v/v) triethylamine in DMF, according to some embodiments. Generally speaking, FIG. 2 illustrates a rhodamine B-hydroxamate based fluorescence detection of diethyl chlorophosphate at various concentrations. Fluorescence emission spectra of the reaction mixtures with addition of diethyl chlorophosphate at 500 ppm, 170 ppm, 100 ppm, 50 ppm, 25 ppm, and 0 ppm (from top to the bottom, respectively) (Ex: 560 nm).

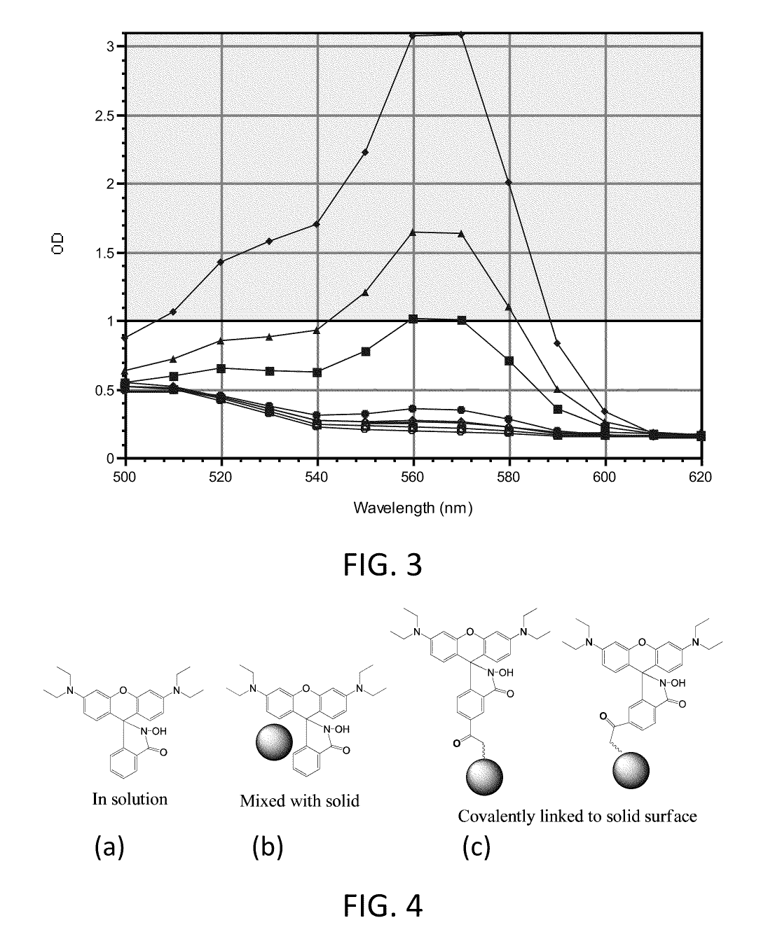

FIG. 3 illustrates UV-Vis absorption spectra of products generated from diethyl chlorophosphate with rhodamine B-hydroxamate (1 mg/mL) in the presence of 3% (v/v) triethylamine in DMF, according to some embodiments. Generally speaking, FIG. 3 describes a rhodamine B-hydroxamate based colorimetric detection of diethyl chlorophosphate at various concentrations, showing UV-Vis absorbance spectra of the reaction mixtures with addition of diethyl chlorophosphate at 500 ppm, 250 ppm, 170 ppm, 100 ppm, 50 ppm, 25 ppm, and 0 ppm (from top to the bottom, respectively)

One of skill will also appreciate that the amounts of nerve agent detected can be determined from signal intensities, whether fluorescence or UV-absorbance, for example. It has been found that, surprisingly, the compounds and methods taught herein provide highly sensitive detection methods for nerve agents. It was observed, for example, that as low as 20 ppm of diethyl chlorophosphate could be easily detected using the compounds and methods provided herein.

One of skill should also appreciate that the methods taught herein are robust. The detection using the hydroxamate can be in solution, mixed with/absorbent in solid support or chemically attached in solid material surface. FIG. 4 illustrates how the sensor can be used (a) in solution, (b) mixed with a solid, or (c) covalently linked to a solid, according to some embodiments. Generally speaking, FIG. 4 illustrates how detection of nerve agents with rhodamine-hydroxamate lactam can be realized in a variety of media including (a) dissolved in organic solution, (b) absorbed on selected solid particles, and (c) covalently attached to a solid surface by select chemical handles that can be installed on rhodamine-hydroxamate lactam, e.g., 5 or 6-carboxy-rhodamine B-hydroxamate.

We claim:

1. A rhodamine-derived detection compound for nerve agents comprising the structure: 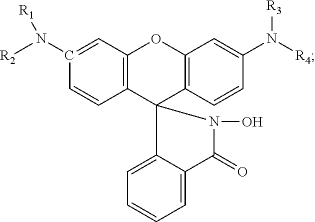
wherein, R₁-R₄ are each independently selected from the group consisting of straight-chain or branched alkylenes having up to 6 carbons and optionally cyclizing with an aromatic host ring; or, a precursor thereof; and,
the compound reacts with a nerve agent to form a fluorescent product for detection of the nerve agent at concentrations below 100 ppm.

2. The compound of claim 1, wherein R₁-R₄ are each independently selected from the group consisting of methylene, ethylene, and propylene.

3. The compound of claim 1, comprising rhodamine B-hydroxamate.

4. The compound of claim 1, wherein the detection compound reacts with a nerve agent to form a fluorescent product for detection of the nerve agent at concentrations as low as about 10 ppm.

5. The compound of claim 1, wherein the nerve agent comprises an organophosphate.

6. The compound of claim 1, wherein the nerve agent is a G-series nerve agent.

7. The compound of claim 1, wherein the nerve agent is a V-series nerve agent.

8. The compound of claim 1, wherein the nerve agent is a Novichok nerve agent.

9. The compound of claim 1, wherein the nerve agent is selected from the group consisting of Sarin, Cyclosarin, Soman, Vx, and Tabun.

10. A rhodamine-derived detection compound for nerve agents, wherein the compound is selected from the group consisting of: 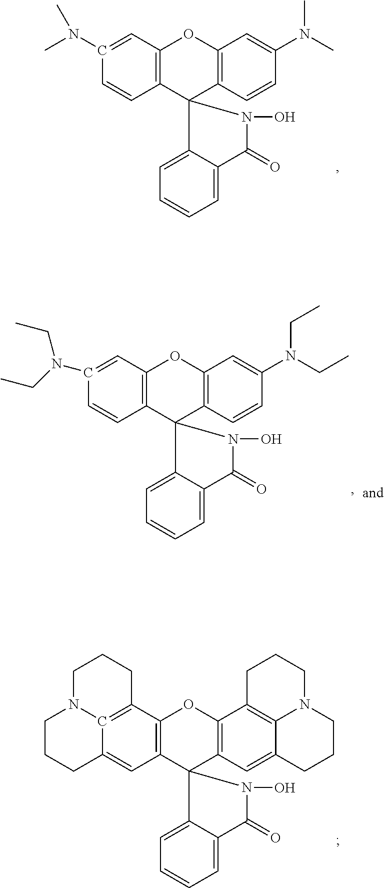
or a precursor thereof.

11. A method of detecting a nerve agent, comprising:
reacting a detection compound having the structure 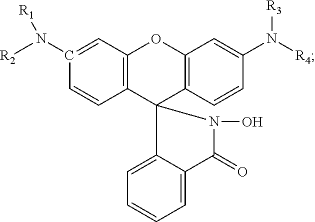
wherein, R₁-R₄ are each independently selected from the group consisting of straight-chain or branched alkylenes having up to 6 carbons and optionally cyclizing with an aromatic host ring;
or, a precursor thereof;
with a nerve agent; and
detecting a fluorescence of a product formed by the reaction;
wherein, the compound reacts with the nerve agent to form a fluorescent product for detection of the nerve agent at concentrations below 100 ppm.

12. The method of claim 11, wherein R₁-R₄ are each independently selected from the group consisting of methylene, ethylene, and propylene.

13. The method of claim 11, wherein the compound comprises rhodamine B-hydroxamate.

14. The method of claim 11, wherein the detection compound reacts with the nerve agent to form a fluorescent product for detection of the nerve agent at concentrations as low as about 10 ppm.

15. The method of claim 11, wherein the nerve agent comprises an organophosphate.

16. The method of claim 11, wherein the nerve agent is a G-series nerve agent.

17. The method of claim 11, wherein the nerve agent is a V-series nerve agent.

18. The method of claim 11, wherein the nerve agent is a Novichok nerve agent.

19. The method of claim 11, wherein the nerve agent is selected from the group consisting of Sarin, Cyclosarin, Soman, Vx, and Tabun.

20. The method of claim 11, wherein the compound is selected from the group consisting of: 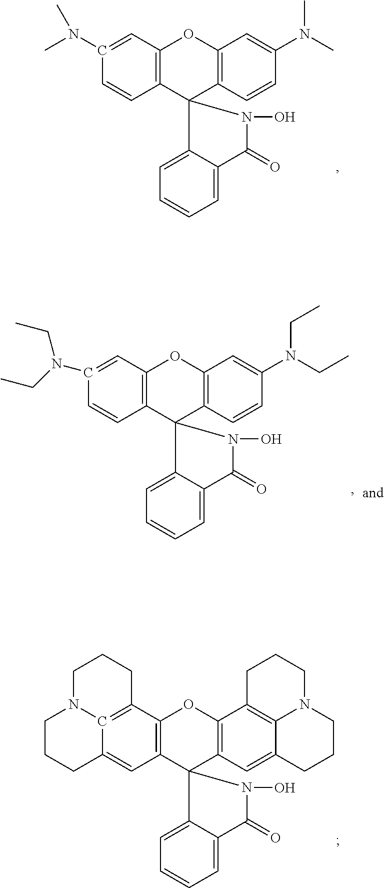
or, a precursor thereof.
